(19) United States
(12) Patent Application Publication
(10) Pub. No.: US 2008/0207942 A1
(43) Pub. Date: Aug. 28, 2008

(54) Chiral Phosphoramidites

(75) Inventors: Manfred T. Reetz, Mulheim an der Ruhr (DE); Gerlinde Mehler, Mulheim an der Ruhr (DE)

Correspondence Address: NORRIS, MCLAUGHLIN & MARCUS, PA, 875 THIRD AVENUE, 18TH FLOOR, NEW YORK, NY, 10022, US

(73) Assignee: Kaiser-Wilhelm-Platz 1, Mulheim an der Ruhr (DE)

(21) Appl. No.: 12/066,790

(22) PCT Filed: Sep. 12, 2006
(86) PCT No.: PCT/DE06/01606

§ 371 Date: Mar. 13, 2008

(30) Foreign Application Priority Data
Sep. 16, 2005 (DE) 10 2005 044 355.9

(51) Int. Cl. C07F 9/547; C07F 9/576; B01J 31/02; C07C 69/34; C07C 229/06

(52) U.S. Cl. 560/155; 568/12; 546/22; 502/167; 560/190

(57) ABSTRACT

Phosphoramidites with the general formulae I to VI are claimed together with the use of these compounds as ligands of transition metal compounds, in particular in transition metal catalysts, in the hydrogenation, transfer hydrogenation, hydroboration, hydrocyanation, 1,4-addition, hydroformylation, hydrosilylation, hydrovinylation, and Heck reactions of prochiral olefins, ketones, or ketimines.

The present invention concerns chiral phosphoramidites with the general formulae II to VI, a procedure for the production of compounds I to VI, chiral transition metal catalysts that contain these phosphoramidites with formulae I to VI and the use of these catalysts in asymmetric transition metal catalysis.

### STATE OF THE TECHNOLOGY

Enantioselective transition metal-catalyzed processes have become increasingly important industrially in the last 30 years, to take transition metal-catalyzed hydrogenation as one example. The ligands required for this are often chiral phosphorus-containing ligands (P-ligands) such as, for example, phosphanes, phosphonites, phosphinites, phosphites, or phosphoramidites, which are bonded with the transition metals, typical examples of which are rhodium, ruthenium, or iridium complexes of optically active diphosphanes such as BINAP.

The development of chiral ligands requires a costly procedure consisting of “design” and “trial and error.” A complementary search method is combinatorial asymmetric catalysis, whereby whole catalogs of modular-structured chiral ligands or catalysts are produced and tested and through which the probability of a find is increased. A disadvantage of all these systems is the relatively considerable expenditure required for preparation to present large numbers of ligands as well as the often inadequate enantioselectivity observed in catalysis. Thus, the goal of industrial and academic research continues to be to produce new, economical, and especially efficient ligands as simply as possible.

While most chiral phosphorus-containing ligands are chelating diphosphorus compounds, especially diphosphanes, which bond with one of the transition metals and stabilize as a chelate complex, thereby determining the degree of asymmetric induction in catalysis, it was recently discovered that some chiral monophosphonites, monophosphites, and monophosphoramidites can also be effective ligands, as, for example, in the case of rhodium-catalyzed asymmetric hydrogenation of prochiral olefins. Well-known examples are BINOL-derived representatives such as the ligands A, B, and C. Spectroscopic and mechanistic studies indicate that two mono-P-ligands are bonded to the metal during catalysis. Hence, the metal-ligand ratio is as a rule 1:2.

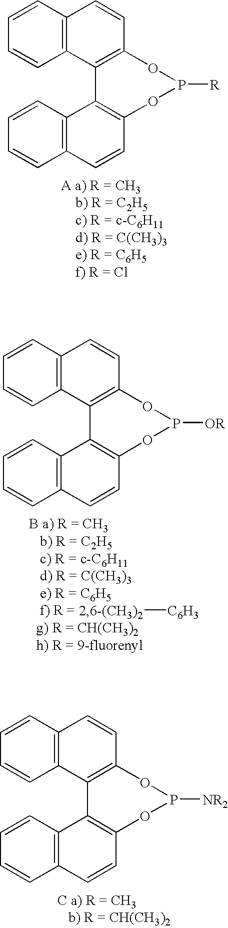

Monophosphorus-containing ligands of types A, B, and C are readily accessible and can be varied quite easily because of their modular structure. By variation of the residues R in A, B, or C, a plethora of chiral ligands can be created, whereby in any given transition metal-catalyzed reaction (e.g., hydrogenation of a prochiral olefin, ketone, or imine or hydroformylation of a prochiral olefin) the ligand can be optimized.

In the international patent application WO 2001094278 A1 chiral monophosphites B, for example, are known ligands for asymmetric transition metal-catalyzed hydrogenation while WO 02/04466 describes the application of the analogous phosphoramidites C. Unfortunately, in this case as well the method has limits, i.e., many of the substrates formed have moderate or poor enantioselectivity, e.g., in hydrogenations or hydroformylations. Therefore, the need for new, economical and effective chiral ligands for industrial application in transition-metal catalysis persists.

Another group of phosphorus-containing ligands, i.e., bidentate phosphoramidites with ethano or propano- bridges are not claimed in WO 02/04466 because of their poor effectiveness. The disclosed compounds contain two BINOL residues on both P atoms that are bridged via diaminoalkyl groups:

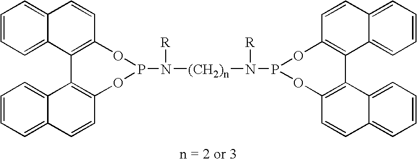

When used as ligands for enantioselective hydrogenation ee-values are obtained that are inadequate for industrial use. Hydrogenation of methyl-2-acetamidocinnamate yields only between 25% and 80% ee for the ethano-bridged and propano-bridged compounds, respectively.

The task of the present invention is hence finding new chiral phosphorus ligands that are simple to produce and can be used as ligands in transition metal-complexes to yield catalysts that are highly efficient in transition metal catalysis, and in particular in hydrogenation, hydroboration and hydrocyanation of olefins, ketones, and ketimes.

Accordingly an object of the present invention is chiral phosphoramidites derived from amines, hydrazines, or diamines with the exception of the familiar ethano-bridged and propano-bridged representatives and, in particular, phosphoramidites with formulae II to VI:

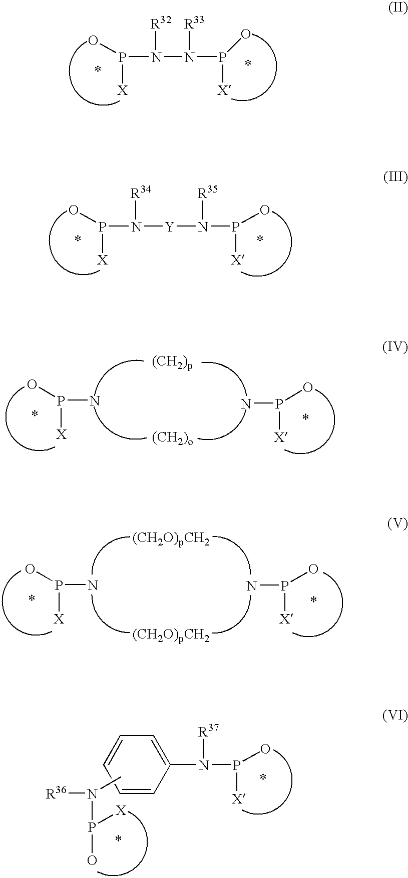

in which

X and X′ may be the same or different and stand for O, S, N—R^{a}, where R^{a}=linear or branched C₁-C₈=alkyl, C₃-C₈-cycloalkyl, aryl or heteroaryl and sulfonyl, Y=(CH₂)ₙ and n is a number from 4 to 10, but preferably 4, 5, 6, 7, 8 or 10 or Y=(CH₂)_{n′}O(CH₂CHRO)ₘ(CH₂)_{n″}, and n′ or n″ are the same or different and are a number from 1 to 3 and m is 0 or 1 and R^{b} is H or CH₃,

p and o can be the same or different and are a number between 1 and 6, but preferably between 1 and 4,

R³², R³³, R³⁴, R³⁵, R³⁶ and R³⁷ stand for C₁-C₁₀-alkyl which can present suitable substituents and

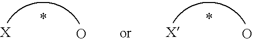

are the same or different and X or X′ stand for O or N—R, i.e., they indicate a component derived from a chiral diol

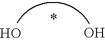

or an amino alcohol

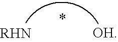

In the preferred variants for the compounds with formula III we have n=3; m=1, R=H; n=3, m=2,R=H; n=3, m=MW=300-1100; R=H or n=3, m=MW 540-4100; R=CH₃.

In the preferred variants of the compounds with formula IV we have n=m=2, n=1, m=2, n=2; m=4, or n=m=3.

In the preferred variants of the compounds with formula V we have n=m=2 or n=2; m=1.

Compounds according to the invention are produced by conversion of the corresponding acid derivatives, preferably acid chlorides, with a diamine or amino alcohol in the presence of a base.

A further object of the invention is accordingly a procedure for the production of chiral phosphoramidites of the general formulae I to VI

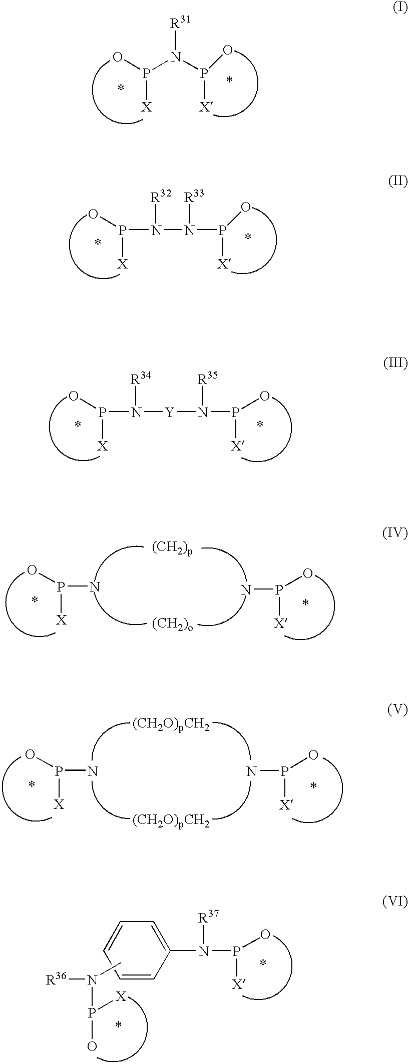

in which

X and X′ may be the same or different and stand for O, S, or N—R^{a} where R^{a}=linear or branched C₁-C₈=alkyl, C₃-C₈-cycloalkyl, aryl or heteroaryl, sulfonyl, Y=(CH₂)ₙ and n is a number from 4 to 10, but preferably 4, 5, 6, 7, 8 or 10 or Y=(CH₂)_{n′}O(CH₂CHRO)ₘ(CH₂)_{n″}, and n′ or n″ are the same or different and are a number from 1 to 3 and m is 0 or 1 and R^{b} is H or CH₃,

p and o can be the same or different and are a number between 1 and 6, but preferably between 1 and 4,

R³¹, R³², R³³, R³⁴, R³⁵, R³⁶ and R³⁷ stand for C₁C₁₀-alkyl which can show suitable substituents and

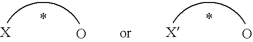

are the same or different and X and X′ stand for O or N—R, i.e., signify a component derived from a chiral diol

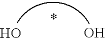

or an amino alcohol

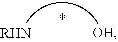

characterized by the fact that the corresponding acid derivative, preferably an acid chloride, reacts with the diamine or amino alcohol in the presence of a base.

The phosphoramidites according to the invention are suited as ligands in transition metal catalysts, especially for hydrogenation, transfer hydrogenation, hydroboration, hydrocyanation, 1,4-addition, hydroformylation, hydrosilylation, hydrovinylation, and Heck reactions of prochiral olefins, ketones, or ketimines.

Another object of the present invention is accordingly transition metal catalysts that disclose transition metal compounds with the above-depicted general formulae I to VI as ligands.

Another object of the present invention is the application of the aforementioned transition metal catalysts in the hydrogenation, transfer hydrogenation, hydroboration, hydrocyanation, 1,4-addition, hydroformylation, hydrosilylation, hydrovinylation, and Heck reactions of prochiral olefins, ketones, or ketimines as well as a procedure for hydrogenation, transfer hydrogenation, hydroboration, hydrocyanation, 1,4-addition, hydroformylation, hydrosilylation, hydrovinylation, and Heck reactions of prochiral olefins, ketones, or ketimines whereby transition metal catalysts are used.

The compounds with the formulae I to VI are derived from dioles or amino alcohols VII to XVI, whereby all enantiomer forms are suitable.

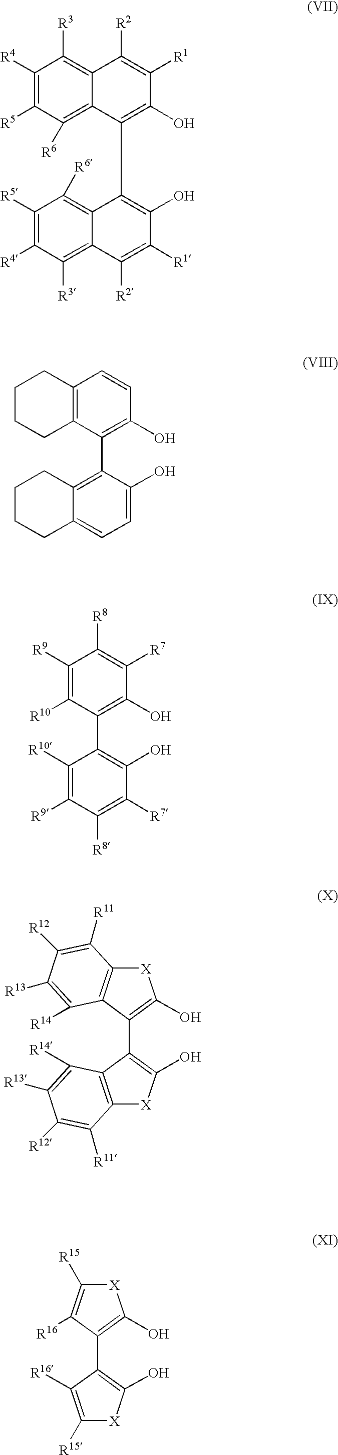

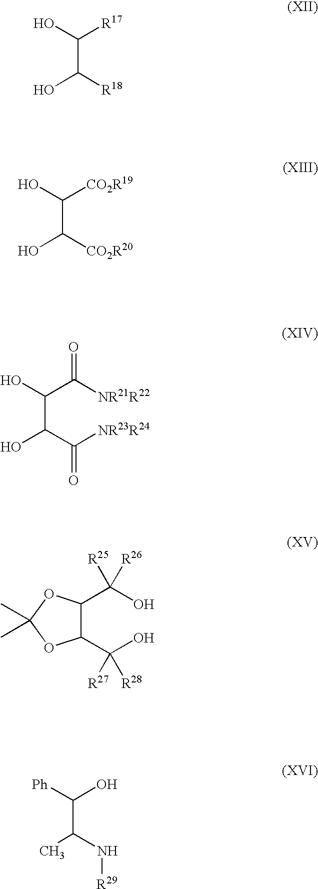

where

R¹, R^{1′}, R², R^{2′}, R³, R^{3′}, R⁴, R^{4′}, R⁵, R^{5′}, R⁶, R^{6′}, R⁷, R^{7′}, R⁸, R^{8′}, R⁹, R^{9′}, R¹⁰, R^{10′}, R¹¹, R^{11′}, R¹², R^{12′}, R¹³, R^{13′}, R¹⁴, R^{14′}, R¹⁵, R^{15′}, R¹⁶, R^{16′}, R¹⁷, R^{17′}, R¹⁸, R¹⁹, R²⁰, R²¹, R²², R²³, R²⁴, R²⁵, R²⁶, R²⁷, R²⁸ and R²⁹ are the same or different and stand for C₁-C₁₀-alkyl, which also may have suitable substituents.

Preferred compounds with the formula XVI are those in which R²⁹ stands for H, C₁-C₆-alkyl, aryl or sulfonyl.

The alkyl residues usually have 1 to 10 carbon atoms and may be either linear or branched. The preferred alkyl residues are those with 1 to 6 carbon atoms such as methyl, ethyl, n-propyl, isopropyl, n-butyl, isobutyl, t-butyl, n-pentyl, iso-pentyl, n-hexyl, iso-hexyl, but also cycloalkyl groups such as cyclopentyl, cyclohexyl, etc. or substituted alkyl groups.

Under the present invention, the aryl or heteroaryl groups used in the ring are aromatic ring systems with 5 to 30 carbon atoms and perhaps also hetero atoms such as N, O, S, P, Si in the ring, whereby the ring may be simple or manifold ring systems. e.g., condensed ring systems or rings bonded together via simple bonds or complex bonds. Examples of aromatic rings are: phenyl, naphthyl, biphenyl, diphenyl ether, diphenylamine, benzophenone, et al. Substituted aryl groups display one or more substituents. Some examples of heteroaryl groups are alkoxyaryl, alkylsulfanyl-substituted alkyl, N-alkylated aminoalkyl, et al. Examples of heteroaryl substituents are: pyrrolyl, pyrrolidinyl, pyridinyl, chinolinyl, indolyl, pyramidinyl, imidazolyl, 1,2,4- triazolyl, tetrazoyl, et al. Examples of heteroatom-containing alicyclic groups are pyrrolidino, morpholino, piperazino, piperidino, etc.

The substituents exhibiting the aforementioned groups are OH, F, Cl, Br, J, CN, NO₂, NO, SO₂, SO₃, amino, acyl, —COOH, —COO(C₁-C₆-alkyl), sulfonyl, mono and di-(C₁-C₂₄-alkyl)-substituted amino-, mono- and di-(C₅-C₂₀-aryl)-substituted amino and imino which can in turn also be substituted, e.g., C₁-C₆-alkyl, aryl and phenyl. The cyclic residues, in particular, may display C₁-C₆ alkyl groups as substituents.

The residues with the general formulae VII to XVI display aryl or heteroaryl residues or functional groups such as cyano, amino, carbonyl residues, sulfonyl or acyl residues as substituents.

Whereas the above ligands with formulae I to VI contain a “backbone” consisting of an amine, hydrazine or diamine, and hence should be called di-phosphoramidites, another object of the invention, the analogous di-phosphorus ligands, whose “backbone” consists of an achiral amino-alcohol

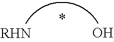

such as

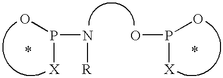

The components of the chiral P-heterocycles are the same as for phosphoramidites derived from hydrazines or diamines. The structure of amino alcohols that function as backbone can vary considerably, as can the nature of the bridge between nitrogen and oxygen as well. (CH₂)ₙ units with, for instance, n=2, 3, 4, 5, 6, 7, 8, 9, or 10 are common. But the bridge can also be of a completely different nature, e.g., a cyclic or aromatic alcohol.

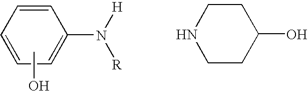

The residues R in the above formulae are preferably alkyl residues with 1 to 6 carbon atoms that may be linear or branched, such as methyl, ethyl, n-propyl, iso-pentyl, n-butyl, isobutyl, n-pentyl, isopentyl, n-hexyl, isohexyl, but also cycloalkyl groups such as cyclopentyl, cyclohexyl, or benzyl. The residues may also be sulfonyl, or aryl or heteroaryl residues such as phenyl, napthyl, or pyridyl.

Most of the chiral phosphoramidite ligands can be quite easily produced by reaction of the corresponding phosphoric acid derivatives, preferably the acid chloride, with a corresponding diamine or amino-alcohol in the presence of a base such as NEt₃. The following reaction equation is an example.

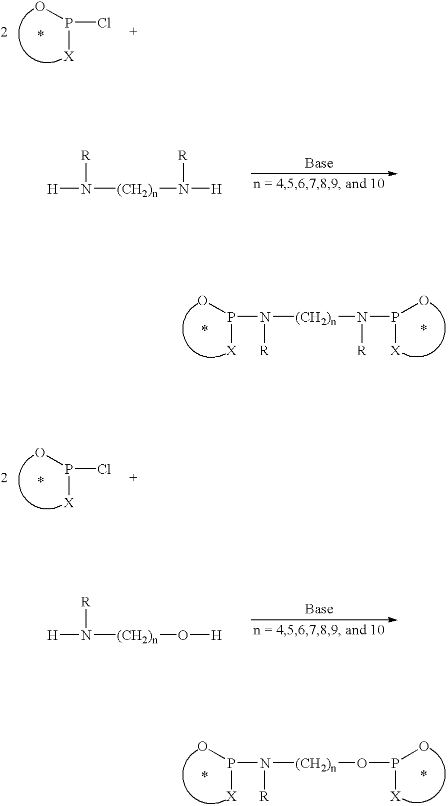

Alternatively, the backbone in the bis-phosphorylated tetrachlor-compound reacts with a chiral diol

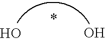

or amino alcohol

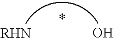

in the presence of a base. This alternative is mainly used in the synthesis of I and II

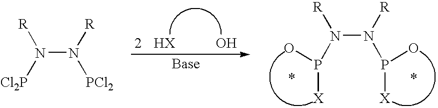

The production of the catalysts or precatalysts may be done with a procedure familiar to professionals. Usually one of the above-described ligands or mixture of ligands is brought together with a suitable transition metal complex. Then an additive such as a phosphine of the type PPh₃ or a phosphite type P(OPh)₃ is added if relevant, along with a N-containing compound such as pyridine or water. The transition metals that may be used are those belonging to the groups IIIb, IVb, Vb, VIb, VIb, VIII, Ib, and IIb of the periodic system as well as lanthanides and actinides. Usually the metals are chosen from among the transition metals of groups VIII and Ib of the periodic system. Specifically, these are transition metal complexes of ruthenium, osmium, cobalt, rhodium, iridium, nickel, palladium, platinum and copper, with preference given to complexes of ruthenium, rhodium, iridium, nickel, palladium, platinum and copper.

The transition metal complexes may be common salts such as MXₙ (X=F, Cl, Br, I, BF₄⁻, BAr₄⁻, where Ar stands for phenyl, benzyl, or 3,5-bis-trifluormethylphenyl, SbF₆⁻, PF₆⁻, ClO₄⁻, RCO₂⁽⁻⁾ CF₃SO₃⁽⁻⁾, Acac⁽⁻⁾), e.g., [Rh(OAc)₂]₂, Rh(acac)₃, Rh(COD)₂BF₄, Cu(CF₃SO₃)₂, CuBF₄, Ag(CF₃SO₃), Au(CO)Cl, In(CF₃SO₃)₃, Fe(ClO₄)₃, NiCl₂(COD) (COD=1.5-cyclooctadiene), Pd(OAc)₂, [C₃H₅PdCl]₂, PdCl₂(CH₃CN)₂ or La(CF₃SO₃)₃, to mention only a few. But it may also be metal complexes that have ligands such as olefins, dienes, pyridine, CO or NO (to mention only a few). The latter are partially or wholly suppressed by the reaction with the P-ligands. Cationic metal complexes may also be used. The professional world knows a multitude of possibilities (G. Wilkinson, Comprehensive Coordination Chemistry, Pergamon Press, Oxford (1987), B. Cornils, W. A. Hermann, Applied Homogenous Catalysis with Organometallic Compounds, VCH, Weinheim (1996)). Common examples are: Rh(COD)₂BF₄, [(Cymol)RuCl₂]₂, (pyridine)₂Ir(COD)BF₄, Ni(COD)₂, (TMEDA)Pd(CH₃)₂ (TMEDA=N, N, N′, N′- tetramethylethylenediamine), Pt(COD)₂, PtCl₂(COD) or [RuCl₂(CO)₃]₂ to mention only a few.

The metal compound and the ligand, i.e., compounds with formulae I to VI, are normally used in such quantities that they form catalytically active compounds. Thus, the quantity of metal compound used can be 25 to 200 mol % relative to the used amount of chiral compound with the general formulae I to VI, whereby 30-100 mol % are preferred, 80-100 ml % are even more preferred and 90 to 100 mol % is most preferred.

Catalysts that contain in situ produced transition metal complexes or isolated transition metal complexes are especially suited for use in a procedure for the production of chiral compounds. Especially preferred are catalysts for asymmetric 1,4 additions, asymmetric hydroformylations, asymmetric hydrocyanations, asymmetric hydroborations, asymmetric hydrosilylation, asymmetric hydrovinylation, asymmetric Heck reactions and asymmetric hydrogenations or transfer hydrogenations.

Another object of the invention is accordingly a procedure for asymmetric transition metal-catalyzed hydrogenation, transfer hydrogenation, hydroboration, hydrocyanation, 1,4-addition, hydroformylation, hydrosilylation, hydrovinylation, and Heck reactions of prochiral olefins, ketones, or ketimines, said procedure being characterized by the fact that the catalysts feature chiral ligands with the aforementioned formulae I to VI.

In a preferred variant of the present invention the transition metal catalysts are used for asymmetric hydrogenation, hydroboration, or hydrocyanation of prochiral olefins, ketones, or ketimines. High yields of the end products are obtained and the optical isomers have a high purity.

The preferred asymmetric hydrogenations or transfer hydrogenations are, for instance, hydrogenations of prochiral C═C bonds as, for instance, prochiral enamines, olefins, and enol ethers, C═O bonds as for example prochiral ketones and C═N bonds as for example prochiral imines. Especially preferred asymmetric hydrogenations are hydrogenations of prochiral enamines and olefins.

The quantity of metal compound or transition metal complex used may be, for instance 0.0001 to 5 mol % relative to the substrate, whereby 0.0001 to 0.5 mol % are preferred, 0.0001 to 0.1 mol % are more preferred and 0.001 to 0.008 mol % are most preferred.

In a preferred variant asymmetric hydrogenations, for instance, can be done in such a way that the catalyst is produced in situ from a metal compound or chiral compound with the general formulae I to VI in a suitable solvent if possible, the substrate is added and the reaction mixture is added at the reaction temperature under hydrogen pressure.

To carry out a hydrogenation, for example, the metal compound and the ligand in a degassed solvent are placed in a heated autoclave. This is agitated for 5 min and then the substrate in a degassed solvent is added. After adjusting the temperature hydrogenation takes place at elevated H₂ pressure.

Examples of suitable solvents for asymmetric hydrogenation are chlorinated alkanes such as methylene chloride, short-chain C₁-C₆-alcohols, such as methanol, iso-propanol or ethanol, aromatic hydrocarbons, such as toluene or benzene, ketones, for example, acetone or carboxylic acid esters such as ethyl acetate.

Asymmetric hydrogenation is done, for example, at a temperature of −20° C. to 200° C., but preferably 0 to 100° C., and especially preferably at 20 to 70° C.

The hydrogen pressure may be 0.1 to 200 bar, 0.5 to 50 is preferred, and 0.5 to 5 bar is especially preferred.

The catalysts according to the invention are especially suited for a procedure for the production of chiral active ingredients of pharmaceutical products and agricultural chemicals or intermediate products of these two classes.

The advantage of the present invention is that good activities with an extraordinary selectivity can be achieved with easy-to-produce ligands, especially in asymmetric hydrogenation.

### EXAMPLES

### Example 1

### Synthesis of 1,6-bis[O,O′-(S)-1,1′-dinaphthyl-2,2′-diyl)-N,N′-dimethylphosphoramidite]hexanediamine

0.44 ml (0.35 g, 2.40mmol) absolute N,N′-dimethyl-1,6-hexane diamine and 0.74 ml (0.535 g, 5.30 mmol) absolute triethylamine were pipetted into 1.69 g (4.80 mmol) (S)-2,2′-binaphthylphosphoric acid ester chloride in 150 ml absolute toluene at room temperature. After a reaction time of 20 h the solid colorless precipitate was filtered off over a D4 fritted filter and washed with 5 ml absolute toluene. The filtrate was then completely freed of the solvent. The crude product thus obtained was purified by column chromatography over silica (70-230 mesh, activity stage 1) (Hexane/dichloromethane - 1:1), yielding 0.64 g (0.83 mmol, 34.5%) of a colorless powder. Analysis: ¹H-NMR (CDCl₃, 300 MHz): 7.96-7.18 (24 H), 3.10(m) [2H], 2.95 (m) [2H], 2.33 (s) [3H], 2.31 (s) [3H], 1.52 (m) [4H], 1.30 (m) [4H]; ³¹P-NMR (CDCl₃, 121 MHz); 149.704; MS (El, evaporation temperature 295° C.); m/z=772 (8.3%), 315 (100%), 112 (88.26%); EA: C: 77.28% (calcd.: 74.60%). H: 5.73% (calcd. 5.47%). P: 7.59% (calcd. 8.01%), N 2.28% (calcd. 3.62%).

### Example 2

### Synthesis of 1,8-bis[O,O′-(S)-1,1′-dinaphthyl-2,2′-diyl)-N,N′-dimethylphosphoramidite]octandiamine

0.145 g (0.84 mmol) absolute N,N′-dimethyl-1,8-octane diamine and 0.26 ml (0.187 g, 1.85 mmol) absolute triethylamine were pipetted at room temperature into 0.59 g (1.68 mmol) (S)-2,2′-binaphthylphosphoric acid ester chloride in 100 ml absolute toluene. After a reaction time of 20 h the solid colorless precipitate was filtered off over a D4 fritted filter and washed with 5 ml absolute toluene. The filtrate was then completely freed of the solvent. The crude product thus obtained was purified by column chromatography over silica (70-230 mesh, activity stage 1) (Hexane/dichlormethane—1:1), yielding 0.12 g (0.15 mmol, 17.8%) of a colorless powder. Analysis: ¹H-NMR (CDCl₃, 300 MHz): 7.88-7.13 [24H], 3.04(m) [2H], 2.88 (m) [2H], 2.25 (s) [3H], 2.23(s) [3H], 1.47 (m) [4H], 1.23 (m) [8H], ³¹P-NMR (CDCl₃, 121 MHz): 149.741; MS (El, evaporation temperature 300° C.): m/z=485 (80.18%), 315 (100%), 268 (42.02%); EA: C: 70.95% (calcd. 74.99%), H: 4.89% (calcd. 5.79%), P: 7.40% (calcd. 7.74%), N: 2.26% (calcd. 3.49%).

### Example 3

### Synthesis of 1,4-bis[O,O′-(S)-1,1′-dinaphthyl-2,2′-diyl)-N,N′-dimethylphosphoramidite]but-2-enediamine

0.150 g (1.30 mmol) absolute N,N′-dimethyl-1,4-but-2-ene diamine and 0.40 ml (0.29 g, 2.87 mmol) absolute triethylamine were added to 0.92 g (2.61 mmol) (S)-2,2′-binaphthylphosphoric acid ester chloride in 100 ml absolute toluene at room temperature. After a reaction time of 20h the solid colorless precipitate was filtered off over a D4 fritted filter and washed with 5 ml absolute toluene. The filtrate was then completely freed of the solvent. The crude product thus obtained was purified by column chromatography over silica (70-230 mesh, activity stage 1) (Hexane/dichloromethane - 1:1), yielding 0.51 g (0.68 mmol, 52.8%) of a colorless powder. Analysis: ¹H-NMR (CDCl₃, 300 MHz): 7.87-7.08 [24H], 5.45 (s) [2H], 3.59 (m) [2H], 3.36 (m) [2H], 2.26(s) [6H]; ³¹P-NMR (CDCl₃, 121 MHz): 149.279; MS (El, evaporation temperature 305° C.): m/z=384 (100%), 315 (13.76%), 268 (19.90%); EA: C: 75.75% (calcd. 74.38%), H: 4.95% (calcd. 4.88%), P: 7.18% (calcd. 8.34%), N: 2.16% (calcd. 3.77%).

### Example 4

### Synthesis of 1,4-bis[O,O′-(S)-1,1′-dinaphthyl-2,2′-diyl) phosphoramidite]diazacyclohexane

0. 172 g (2.00 mmol) piperazine and 0.62 ml (0.44 g, 4.40 mmol) absolute triethylamine were added to 1.40 g (4.00 mmol) (S)-2,2′-binaphthylphosphoric acid ester chloride in 100 ml absolute toluene at room temperature. After a reaction time of 20 h the solid colorless precipitate was filtered off over a D4 fritted filter and washed with 5 ml absolute toluene. The filtrate was then completely freed of the solvent, yielding after recrystallization from dichloromethane 0.90 g (1.26 mmol, 63.0%) of a colorless powder. Analysis: ¹H-NMR (CDCl₃, 300 MHz): 7.94-7.02 [24H], 2.75 (s) [8H]; ³¹P-NMR (CDCl₃, 121 MHz): 145.371; MS (El, evaporation temperature 340° C.): m/z=714 (34.53%), 315 (100%), 268 (59.20%); EA: C: 75.35% (calcd. 73.94%), H: 4.45% (calcd. 4.51%), P: 8.98% (calcd. 8.66%), N: 3.83% (calcd. 3.91%).

### Example 5

### Synthesis of 1,4-bis[O,O′-(S)-1,1′-dinaphthyl-2,2′-diyl)-phosphoramidite]diazacycloheptane

0.20 g (1.96 mmol) homopiperazine and 0.60 ml (0.43 g, 4.32 mmol) absolute triethylamine were added to 1.38 g (3.93 mmol) (S)-2,2′-binaphthylphosphoric acid ester chloride in 100 ml absolute toluene at room temperature. After a reaction time of 20 h the solid colorless precipitate was filtered off over a D4 fritted filter and washed with 5 ml absolute toluene. The filtrate was then completely freed of the solvent. The crude product thus obtained was purified by column chromatography over silica (70-230 mesh, activity stage 1) (Hexane/dichloromethane—1:1), yielding 0.38 g (0.52 mmol, 26.6%) of a colorless powder. Analysis: ¹H-NMR (CDCl₃, 300 MHz): 7.90-7.15 [24H], 3.05 (m) [4H], 2.91 (m) [4H], 1.51 (m) [2H]; ³¹P-NMR (CDCl₃, 121 MHz): 148.869; MS (El, evaporation temperature 288° C.): m/z=413 (100%), 315 (60.66%), 268 (33.16%); EA: C: 73.86% (calcd. 74.17), H: 5.56% (calcd. 4.70%), P: 7.65% (calcd. 8.50%), N: 3.05% (calcd. 3.84%).

### Example 6

### Synthesis of 4-bis[O,O′-(S)-1,1′-dinaphthyl-2,2′-diyl)-phosphoramidite]piperidine

0.16 g (1.63 mmol) 4-hydroxypiperidine and 0.44 ml (0.36 g, 3.60 mmol) absolute triethylamine were added to 1.14 g (3.25 mmol) (S)-2,2′-binaphthylphosphoric acid ester chloride in 100 ml absolute toluene at room temperature. After a reaction time of 20 h the solid colorless precipitate was filtered off over a D4 fritted filter and washed with 5 ml absolute toluene. The filtrate was then completely freed of the solvent. The crude product thus obtained was purified by column chromatography over silica (70-230 mesh, activity stage 1) (Hexane/dichloromethane—1:1), yielding 0.19 g (0.26 mmol, 15.9%) of a colorless powder. Analysis: ¹H-NMR (CDCl₃, 300 MHz): 7.88-7.10 [24H], 3.17 (m) [4H], 2.63 (m) [4H], 1.70 (m) [2H], 1.52 (m) [2H], 1.15 (m) [1H]; ³¹P-NMR (CDCl₃, 121 MHz): 145.301(d) J=53 Hz; MS (El, evaporation temperature 295° C.): m/z=397 (94.88%), 315 (100%), 268 (60.53%); EA: C: 75.47% (calcd. 74.07%), H: 4.92% (calcd. 4.55%), P 8.06% (calcd. 8.49%), N: 1.12% (calcd. 1.91%).

### Example 7

### Synthesis of 3-bis[O,O′-(S)-1,1′-dinaphthyl-2,2′-diyl)-phosphoramidite]piperidine

0.096 g (0.95 mmol) 3-hydroxypiperadine and 0.29 ml (0.21 g, 2.08 mmol) absolute triethylamine were added to 0.66 g (1.90 mmol) (S)-2,2′-binaphthylphosphoric acid ester chloride in 100 ml absolute toluene at room temperature. After a reaction time of 20 h the solid colorless precipitate was filtered off over a D4 fritted filter and washed with 5 ml absolute toluene. The filtrate was then completely freed of the solvent. The crude product thus obtained was purified by column chromatography over silica (70-230 mesh, activity stage 1) (Hexane/dichloromethane—1:1), yielding 0.19 g (0.26 mmol, 27.4%) of a colorless powder. Analysis: ¹H-NMR (CDCl₃, 300 MHz): 7.90-7.15 [24H], 2.98 (m) [1H], 2.85 (m) [2H], 2.62 (m) [2H], 1.32 (m) [2H], 1.21 (m) [2H]; ³¹P-NMR (CDCl₃, 121 MHz): 146.271 (d) J=84 Hz; MS (El, evaporation temperature 265° C.): m/z=398 (100%), 315 (90.90%), 268 (67.11%); EA: C: 74.37% (calcd. 74.07), H: 5.13% (calcd. 4.55%), P: 7.69% (calcd. 8.49%), N: 1.58% (calcd. 1.91%).

### Example 8

### Synthesis of 1,4-bis[O,O′(S)-1,1′-dinaphthyl-2,2′-diyl)-phosphoramidite]-4,10-diaza-15-crown-5

1.43 ml (1.03 g, 10.24 mmol) absolute triethylamine was added to 1.1 ml (1.76 g, 12.80 mmol) phosphortrichloride in 50 ml toluene at room temperature. Then 0.56 (2.55 mmol) 4,10-diaza-15-crown-5 was dissolved in 30 ml toluene and added to the above by drops over a period of 1 h at room temperature. After a reaction time of 20 h the solid colorless precipitate was filtered off over a D4 fritted filter and washed with 5 ml absolute toluene. The filtrate was then completely freed of the solvent. The residue thus obtained was dissolved in 30 ml toluene and 1.57 ml (1.14 g, 11.26 mmol) trimethylamine was added. Then 1.47 g (5.12 mmol) (S)-2,2′-binaphthol was dissolved in 50 ml toluene and added by drops over a period 15 min. After a reaction time of 20 h the solid colorless precipitate was filtered off over a D4 fritted filter and washed with 5 ml absolute toluene. The filtrate was then completely freed of the solvent. The resultant yield was 2.10 g (2.48 mmol, 96.8%) of a colorless powder. Analysis: ¹H-NMR (CDC₂Cl₂, 300 MHz): 7.91-7.13 [24H], 3.41(m) [12H], 3.06 (m) [8H]; ³¹P-NMR (CDCl₃, 121 MHz): 150.321; EA: C: 72.96% (calcd. 70.91), H: 5.90% (calcd. 5.23%), P: 6.08% (calcd. 7.31%), N: 2.69% (calcd. 3.30%).

### Example 9

### Synthesis of 1,4-bis[O,O′(S)-1,1′-dinaphthyl-2,2′-diyl)-phosphoramidite]-4,13-diaza-18-crown-6

1.43 ml (1.03 g, 10.24 mmol) absolute triethylamine was added to 1.1 ml (1.76 g, 12.80 mmol) phosphortrichloride in 50 ml toluene at room temperature. Then 0.67 g (2.55 mmol) 4,13-diaza-18-crown-6 was dissolved in 30 ml toluene and added to the above by drops over a period of 1 h at room temperature. After a reaction time of 20 h the solid colorless precipitate was filtered off over a D4 fritted filter and washed with 5 ml absolute toluene. The filtrate was then completely freed of the solvent. The residue thus obtained was dissolved in toluene and 1.57 ml (1.14 g, 11.26 mmol) triethylamine was added. Then 1.47 g (5.12 mmol) (S)-2,2′-binaphthol was dissolved in 50 ml toluene and added by drops over a period 15 min. After a reaction time of 20h the solid colorless precipitate was filtered off over a D4 fritted filter and washed with 5 ml absolute toluene. The filtrate was then completely freed of the solvent. The resultant yield was 1.74 g (1.95 mmol, 76.6%) of a colorless powder. Analysis: ¹H-NMR (CD₂Cl₂, 300 MHz): 7.94-7.01 [24H], 3.36(m) [16H], 3.03 (m) [8H], ³¹P-NMR (CDCl₃, 121 MHz): 150.11; MS (ESI soln.: CH₂CL₂, pos. ions) MG 890; EA: C: 72.16% (calcd. 70.10), H: 6.10% (calcd. 5.43), P: 5.80% (calcd. 6.95%), N: 2.59% (calcd. 3.14%).

### Example 10

### Synthesis of (S,S)-dinaphtho[2,1-d:1′,2′-f][1,3,2)-dioxaphosphepin-1,2-dimethyl hydrazine

0.73 g (2.88 mmol) 1,2-bis(dichlorophosphino)-1,2-dimethyl hydrazine and 1.6 ml (1.20 g, 11.80 mmol) absolute triethylamine at −80 C were added to 1.59 g (5.57 mmol) (R)-2,2′binaphthol resting at room temperature in 100 ml toluene. After a reaction time of 20 h at room temperature the solid colorless precipitate was filtered off over a D4 fritted filter and washed with 5 ml absolute toluene. The filtrate was than completely freed of the solvent. The crude product thus obtained was purified by column chromatography over silica (70-230 mesh, activity stage 1) (Hexane/dichloromethane—1:1), yielding 0.28 g (0.40 mmol, 14.1%) of a colorless powder. Analysis: ¹H-NMR (CDCl₃, 300 MHz): (2 diastereomers 60:40) 7.95-7.15 [24H], 2.63 (s) [3H], 2.35 (s) [3H]; ³¹P-NMR (CDCl₃, 121 MHz): 147.218, 142.945; MS (El, evaporation temperature 300° C.): m/z=688 (15.94%), 315 (100%), 268 (34.53%); EA: C: 68.12% (calcd. 73.25%), H: 4.60% (calcd. 4.39%), P: 7.84% (calcd. 8.99%), N: 3.42% (calcd. 4.06%).

### Presentation of Metal Complexes

### Example 11

### Synthesis of (η²,η²-cycloocta-1,5-dien-1,4-bis[O,O′-(S)-1,1′-dinaphthyl-2,2′-diyl)-phosphoramidite]-diazacyclohexane-rhodium(I)-tetrafluoroborate

42.6 mg (59.6 μmol) 1,4-bis[O,O′]-(S)-1,1′-dinaphthyl-2,2′ diyl)phosphoramidite]-diazacyclohexane (ligand 4) and 24.2 mg (59.6 μmol) Bis-(1,5-cyclooctadien)-rhodium(I)-tetrafluoroborate were agitated at room temperature in 5 ml absolute dichloromethane for 20 hours. The orange-colored solution was then washed completely free of solvent, yielding a reddish-orange powder. Analysis: ³¹P-NMR (CD₂Cl₂, 121 MHz): 140.8 (m,J_{P,P}=42 Hz, J_{Rh,P}=243 Hz), 133.1 (m,J_{P,P}=41 Hz, J_{Rh,P}=240 Hz).

### Example 12

### Synthesis of (η²,η²-cycloocta-1,5-dien-1,4-bis[O,O′-(S)-1,1′-dinaphthyl-2,2′-diyl)-phosphoramidite]-diazacycloheptane-rhodium(I)tetrafluoroborate

31.7 mg (43.5 μmol) 1,4-bis[O,O′-(S)-1,1′-dinaphthyl-2,2′-diyl)-phosphoramidite]diazacycloheptane (ligand 5) and 17.7 mg (43.5 μmol) Bis-(1,5-cyclooctadien)-rhodium(I)-tetrafluoroborate were agitated at room temperature in 5 ml absolute dichloromethane for 20 hours. The orange-colored solution was then washed completely free of solvent, yielding a reddish-orange powder. Analysis: ³¹P-NMR (CD₂Cl₂, 121 MHz): 140.8 (m), 133.1(m).

### Example 13

### Synthesis of (η²,η²-cycloocta-1,5-dien-(S)-dinaphtho[2,1-d:1′,2′-f][1,3,2]dioxaphosphepine-4-amine-rhodium (I)-tetrafluoroborate

27.8 mg (40.4 μmol) (S)-dinaphtho [2,1-d:1′,2′-f][1,3,2]dioxaphosphepine-4-amine (ligand 8) and 16.4 mg (40.4 μmol) Bis-(1,5-cyclooctadien)-rhodium(I)-tetrafluoroborate were agitated at room temperature in 5 ml absolute dichloromethane for 20 hours. The orange-colored solution was then washed completely free of solvent, yielding a reddish-orange powder. Analysis: ³¹P-NMR (CD₂Cl₂, 121 MHz): 156.190 (d, ¹J_{RhP}=249 Hz).

### Example 14

### Synthesis of (η²,η²-cycloocta-1,5-dien-(S,S)-dinaphtho[2,1-d: 1′,2′-f][1,3,2]dioxaphosphepine-1,2-dimethyl hydrazine-rhodium(I)-tetrafluoroborate

18.7 mg (25.9 μmol) (S,S)-dinaphtho [2,1-d:1′,2′f][1,3,2]dioxaphosphepine-1,2-dimethyl hydrazine (ligand 10) and 10.5 mg (25.9 μmol) Bis(1,5-cyclooctadien)-rhodium (I)-tetrafluoroborate were agitated at room temperature in 5 ml absolute dichloromethane for 20 hours. The orange-colored solution was then washed completely free of solvent, yielding a reddish-orange powder. Analysis: ³¹P-NMR (CD₂Cl₂, 121 MHz): 120.158 (d, ¹J_{RhP}=199 Hz), 100.26 (d, ¹J_{RhP}=234 Hz. [sic.]

### Hydrogenations

General Procedure for Hydrogenation with in Situ Produced Catalyst

0.5 ml of a 2 mM solution of [Rh(cod)₂]BF₄ in dichloromethane was placed in a round flask equipped with a side tap. Then 0.5 ml of a 2 mM solution of the indicated ligands was added followed by 9.0 ml of a 0.11M substrate solution in dichloromethane. The solution was then saturated with hydrogen and agitated for 20 h at 1.3 bar hydrogen pressure. 2 ml of the resultant solution was filtered over silica (70-230 mesh, activity stage I) and analyzed by gas chromatography.

### Examples 15-24

### Enantioselective Hydrogenation of Dimethylitaconate

Examples 15-24 describe hydrogenation of the substrate dimethylitaconate to 2-methylsuccinic acid dimethyl ester in accordance with the “General procedure for hydrogenation with in situ produced catalyst.” The precise reaction conditions and the yields obtained as well as enantioselective activities are given in Table 1.

TABLE 1

| | | Ligand L | Yield | ee |
|---|---|---|---|---|
| Example | Config. | from ex. | in %^{[a]} | in % |
| 15 | (S) | 1 | 100 | 93.2 (S) |
| 16 | (S) | 2 | 100 | 90.4 (S) |
| 17 | (S) | 3 | 74.1 | 77.8 (S) |
| 18 | (S) | 4 | 100 | 96.4 (S) |
| 19 | (S) | 5 | 100 | 99.2 (S) |
| 20 | (S) | 6 | 100 | 95.6 (S) |
| 21 | (S) | 7 | 100 | 91.0 (S) |
| 22 | (S) | 8 | 100 | 81.1 (S) |
| 23 | (R) | 9 | 100 | 81.8 (R) |
| 24 | (S) | 10 | 100 | 99.6 (S) |

^{[a]}If no further educt was detectable by gas chromatography, the yield is 100%

### Examples 25-34

### Enantioselective Hydrogenation of 2-Acetamidoacrylic acid methylester

Examples 25-34 describe the hydrogenation of the substrate 2-acetamidoacrylic acid methylester to N-acetylalanine methylester in accordance with the “General procedure for hydrogenation with in situ produced catalyst.” The precise reaction conditions and the yields obtained as well as enantioselective activities are given in Table 2.

TABLE 2

| | | Ligand L | Yield | ee |
|---|---|---|---|---|
| Example | Config. | from ex. | in %^{[a]} | in % |
| 25 | (S) | 1 | 100 | 96.0 (S) |
| 26 | (S) | 2 | 100 | 98.0 (S) |
| 27 | (S) | 3 | 85.3 | 88.4 (S) |
| 28 | (S) | 4 | 100 | 99.0 (S) |
| 29 | (S) | 5 | 100 | 99.2 (S) |
| 30 | (S) | 6 | 100 | 94.8 (S) |
| 31 | (S) | 7 | 100 | 92.2 (S) |
| 32 | (S) | 8 | 80.9 | 68.8 (S) |
| 33 | (R) | 9 | 84.4 | 71.0 (R) |
| 34 | (S) | 10 | 100 | 81.1 (S) |

^{[a]}If no further educt was detectable by gas chromatography, the yield is 100%

1. A chiral phosphoramidite of the formulae II-VI: 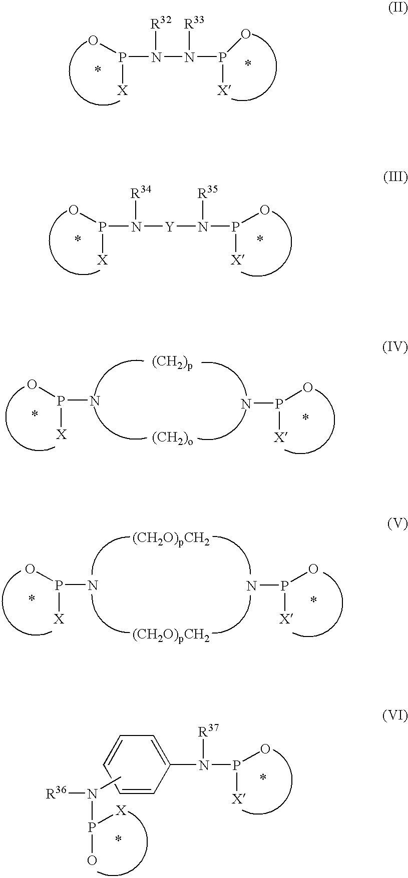
in which
X and X′ may be the same or different and stand for O, S, N—R^{a}, where R^{a}=linear or branched C₁-C₈-alkyl, C₃C₈-cycloalkyl, aryl or heteroaryl and sulfonyl, Y=(CH₂)ₙ and n is a number from 4 to 10, or Y=(CH₂)ₙO(CH₂CHRO)ₘ(CH₂)_{n″}, and n′ or n″ are the same or different and are a number from 1 to 3 and m is 0 or 1 and R^{b} is H or CH₃, p and o can be the same or different and are a number between 1 and 6,
R³², R³³, R³⁴, R³⁵, R³⁶ and R³⁷ stand for C₁-C₁₀-alkyl which is optionally substituted and 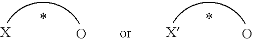
are the same or different and X or X′ stand for O or N—R, i.e., they indicate a component derived from a chiral diol 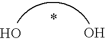
or an amino alcohol

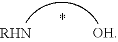

2. The chiral phosphoramidite according to claim 1, wherein the chiral residues 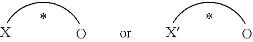
are selected from compounds with the formulae VII-XVI: 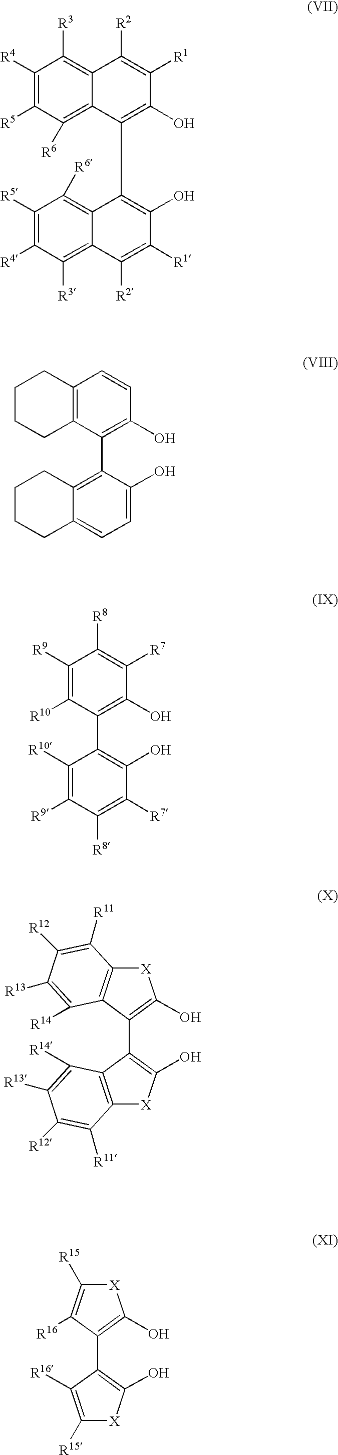 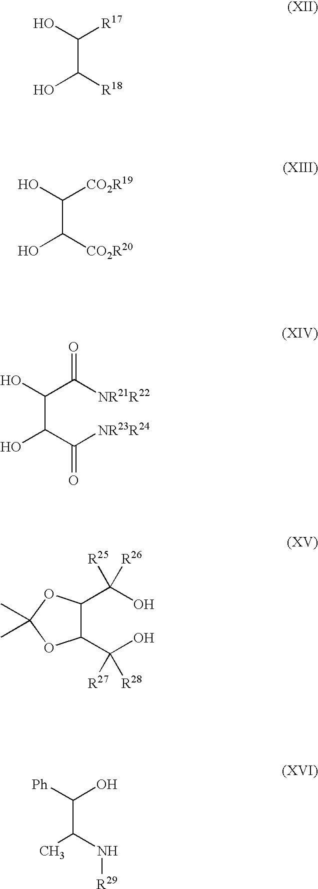
R¹, R^{1′}, R², R^{2′}, R³, R^{3′}, R⁴, R^{4′}, R⁵, R^{5′}, R⁶, R^{6′}, R⁷, R^{7′}, R⁸, R^{8′}, R⁹, R^{9′}, R¹⁰, R^{10′}, R¹¹, R^{11′}, R¹², R^{12′}, R¹³, R^{13′}, R¹⁴, R^{14′}, R¹⁵, R^{15′}, R¹⁶, R^{16′}, R¹⁷, R^{17′}, R¹⁸, R¹⁹, R²⁰, R²¹, R²², R²³, R²⁴, R²⁵, R²⁶, R²⁷, R²⁸ and R²⁹ are the same or different and stand for C₁C₁₀-alkyl, which is optionally substituted by aryl or heteroaryl residues, or aryl or heteroaryl residues or sulfonyl or acyl residues.

3. The chiral phosphoramidite according to claim 1, wherein in compounds with formula III: n=3; m=1; R=H; n=3; m=2; R=H; n=3; M=MW 300-1100, R=H or n=3; m=MW 540-4100; and R=CH₃.

4. The chiral phosphoramidite according to claim 1, wherein in compounds with formula IV: n=m=2, n=1; m=2, n=2; m=4 or n=m=3.

5. The chiral phosphoramidite according to claim 1, wherein in compounds with formula V: n=m=2 or n=2; m=1.

6. A process for producing a chiral phosphoramidite of the formulae I-VI: 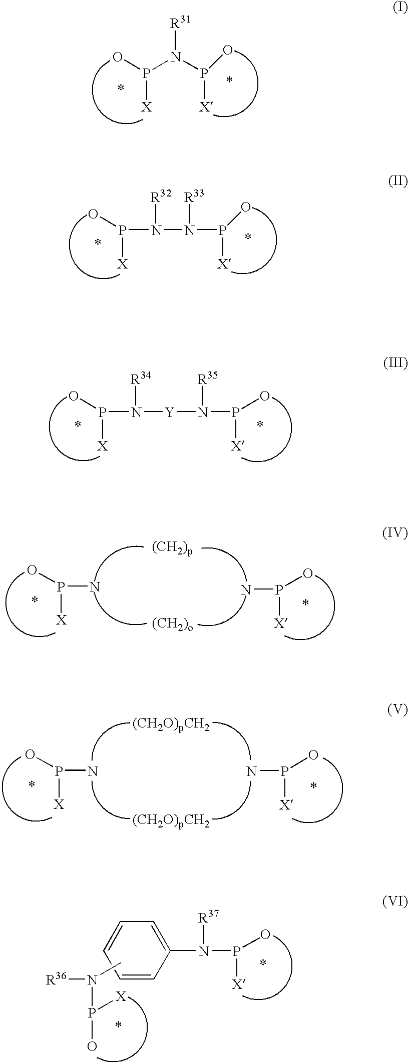
where
X and X′ may be the same or different and stand for O, S, or N—R^{a} where R^{a}=linear or branched C₁-C₈-alkyl, C₃C₈-cycloalkyl, aryl or heteroaryl, sulfonyl,
Y=(CH₂)ₙ and n is a number from 4 to 10, Y=(CH₂)_{n′}O(CH₂CHRO)ₘ(CH₂)_{n″} and n′ or n″ are the same or different and are a number from 1 to 3, and m is 0 or 1 and R^{b} is H or CH₃, and p and o can be the same or different and are a number between 1 and 6,
R³¹, R³², R³³, R³⁴, R³⁵, R³⁶ and R³⁷ stand for C₁C₁₀-alkyl which is optionally substituted and 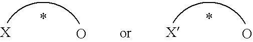
are the same or different and X and X′ stand for O or N—R, i.e., signify a component derived from a chiral diol 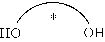
or an amino alcohol 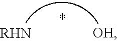
said process comprising reacting the corresponding acid derivative, with the diamine or amino alcohol in the presence of a base.

7. A transition metal catalyst comprising at least one chiral compound of the formulae I to VI: 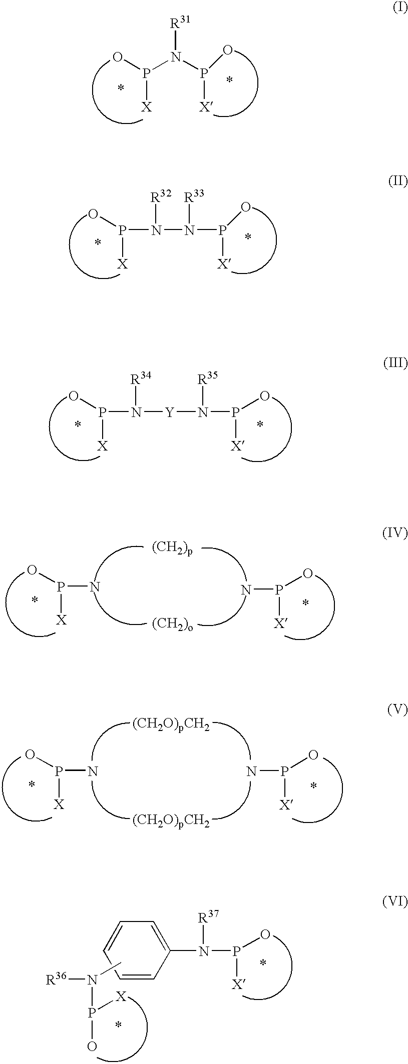
in which
X and X′ may be the same or different and stand for O, S, or N—R^{a} where R^{a}=linear or branched C₁-C₈-alkyl, C₃C₈-cycloalkyl, aryl or heteroaryl, sulfonyl,
Y=(CH₂)ₙ and n is a number from 4 to 10, or Y=(CH₂)_{n′}O(CH₂CHRO)ₘ(CH₂)_{n″}, and n′ or n″ are the same or different and are a number from 1 to 3, and m is 0 or 1 and R^{b} is H or CH₃, and p and o can be the same or different and are a number between 1 and 6,
R³¹, R³², R³³, R³⁴, R³⁵, R³⁶ and R³⁷ stand for C₁C₁₀-alkyl which is optionally substituted and 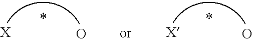
are the same or different and X and X′ stand for O or N—R, i.e., signify a component derived from a chiral diol 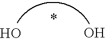
or an amino alcohol

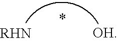

8. A process for producting a transition metal catalyst comprising a transition metal complex of at least one chiral compound having the formulae I to VI, said process comprising reacting at least one transition metal salt with a chiral compound having the formulae I to VI.

9. Process according to claim 8 wherein the transition metal salts are selected from among the groups IIIb, IVb, Vb, VIb, VIIb, VIII, Ib, and IIb of the periodic system as well as lanthanides and actinides.

10. A process comprising asymmetric transition metal-catalyzed hydrogenation, transfer hydrogenation, hydroboration, hydrocyanation, 1,4-addition, hydroformylation, hydrosilylation, hydrovinylation, or a hook-on reaction of prochiral olefins, ketones, or ketimines, wherein the process is carried out in the presence of at least one catalyst having chiral ligands with the formulae I-VI.

11. Process according to claim 10, wherein the catalyst comprises a complex comprising an anion selected from the group consisting of BF₄⁻, BAr₄⁻, SbF₆⁻, and PF₆⁻ where Ar stands for phenyl, pentafluorophenyl, benzyl, or 3,5-bis-trifluormethylphenyl.

12. Process according to claim 6. which further comprises choosing a prochiral preliminary stage from among olefins, ketones, or ketimines and subjecting to hydrogenation, transfer hydrogenation, hydroboration, hydrocyanation, 1,4-addition, hydroformylation, hydrosilylation, hydrovinylation, and a hook-on reaction in the presence of a transition metal catalyst, wherein the transition metal catalyst has chiral ligands selected from compounds having the formulae I-VI.
